(19) United States
(12) Patent Application Publication
(10) Pub. No.: US 2005/0065437 A1
(43) Pub. Date: Mar. 24, 2005

(54) Medical device with markers for magnetic resonance visibility

(75) Inventors: Jan Weber, Maple Grove, MN (US); Timothy J. Ley, Shoreview, MN (US)

Correspondence Address: Todd R. Fronek;Westman, Champlin & Kelly, Suite 1600, 900 Second Avenue South, Minneapolis, MN, 55402-3319, US

(73) Assignee: SciMed Life Systems, Inc., Maple Grove, MN (US)

(21) Appl. No.: 10/670,433

(22) Filed: Sep. 24, 2003

(51) Int. Cl. A61F 2/06

(52) U.S. Cl. 600/431; 623/1.34

(57) ABSTRACT

Embodiments of the present invention relate to medical devices that provide a suitable disturbance of medical resonance images taken of the devices in order to enhance the visibility of the devices. In one embodiment, paramagnetic and/or ferromagnetic material is applied to a support structure of a device. The magnetic materials provide a suitable distortion of MRI images such that the device (or portions thereof) are easily visible and detectable.

### BACKGROUND OF THE INVENTION

The present invention relates generally to devices for use in vascular treatments. More particularly, the present invention relates to devices used in vascular treatments that enhance magnetic resonance visibility, the devices being adapted for use in magnetic resonance imaging.

Vascular stents are known medical devices used in various vascular treatments of patients. Stents commonly include a tubular member that is moveable from a collapsed, low profile, delivery configuration to an expanded, deployed configuration. In the expanded configuration, an outer periphery of the stent frictionally engages an inner periphery of a lumen. The deployed stent then maintains the lumen such that it is substantially unoccluded and flow there through is substantially unrestricted. However, various stent materials and designs render the stent substantially invisible during a Magnetic Resonance Imaging procedure.

A guide wire is used to deliver stents and other devices to positions within the body for various purposes. In many instances, the guide wires are made of a polymer, ceramic or combinations thereof. While polymer and ceramic guide wires provide adequate flexibility to guide devices throughout the body, they are difficult to view during Magnetic Resonance Imaging procedures.

Magnetic Resonance Imaging (MRI) is a non-invasive medical procedure that utilizes magnets and radio waves to produce a picture of the inside of a body. An MRI scanner is capable of producing pictures of the inside of a body without exposing the body to ionizing radiation (X-rays). In addition, MRI scans can see through bone and provide detailed pictures of soft body tissues.

A typical MRI scanner includes a magnet that is utilized to create a strong homogeneous magnetic field. A patient is placed into or proximate the magnet. The magnetic field causes a small majority of the atoms with a net magnetic moment, also referred to as spin, to align in the same direction as the magnetic field. When a radiowave is directed at the patient's body, atoms precessing in the magnetic field with a frequency equal to the radiowave are able to adapt the radiowave energy, which causes them to “tumble over” and align in the opposite direction of the magnetic field. The frequency at which atoms with a net spin precess in a magnetic field is also referred to as the Larmor frequency.

The opposing alignment is at a higher energy level compared to the original orientation. Therefore, after removing the radiowave, atoms will return to the lower energetic state. As the atoms return to the lower energetic state, a radio signal is emitted at the Larmor frequency. These return radio waves create signals (resonance signals) that are detected by the scanner at numerous angles around the patient's body. The signals are sent to a computer that processes the information and compiles an image or images. Typically, although not necessarily, the images are in the form of 2-dimensional “slice” images.

Distortion of these images is generally due to two effects. The first effect is due to magnetic susceptibility of materials subject to the MR imaging. Materials with high magnetic susceptibility generally will distort the images such that the material is readily visible in an MRI procedure. Another effect that distorts images is associated with Faraday's Law. Faraday's Law simply states that any change in a magnetic environment of a coil will cause a voltage to be “induced” in the coil. The induced voltage counteracts the magnetic flux through the coil. During an MRI procedure, when a magnetic field is induced proximate an electrical loop, images taken proximate the loop are distorted and consequently provide poor MR images.

Many stents today are made of a variety of materials. These stents include balloon expandable stents that are made out of Nitinol, Tantalum, Titanium, Niobium and other low magnetic susceptibility alloys. Stent materials also include polymers and ceramics that may be used alone or in combination with a variety of materials including metallic materials. In stent designs where RF artifacts (disturbances in the magnetic fields) are absent during an MRI procedure because there are no electrical loops in the structure and the MRI visibility of the stent depends on a disturbance created by the magnetic susceptibility of the material of the stent, stents made of materials of low magnetic susceptibility posses poor visibility.

An ability to effectively view polymer guide wires and stents during an MRI procedure is desirable. In particular, viewing the guide wire and stent is desirable both during deployment and after deployment of the stent in a patient to evaluate and monitor the operation of the stent.

### SUMMARY OF THE INVENTION

Embodiments of the present invention relate to medical devices that provide a suitable disturbance of medical resonance images taken of the devices in order to enhance the visibility of the devices. In one embodiment, paramagnetic and/or ferromagnetic material is coated onto a base material of a support structure. The coating provides a suitable distortion of MRI images such that the support structure (or portions thereof) are easily visible and detectable.

### BRIEF DESCRIPTION OF THE DRAWINGS

FIG. 1 is a partial block diagram of an illustrative magnetic resonance imaging system.

FIG. 2 is a view of a stent according to one embodiment of the present invention.

FIG. 3 illustrates an exemplary environment for performing plasma immersion ion implantation on a stent.

FIG. 4 illustrates a stent wherein only end portions of the stent include magnetic materials.

FIG. 5 illustrates an exemplary environment for performing plasma immersion ion implantation on a portion of a stent.

FIG. 6 illustrates a guide wire according to an embodiment of the present invention.

### DETAILED DESCRIPTION OF ILLUSTRATIVE EMBODIMENTS

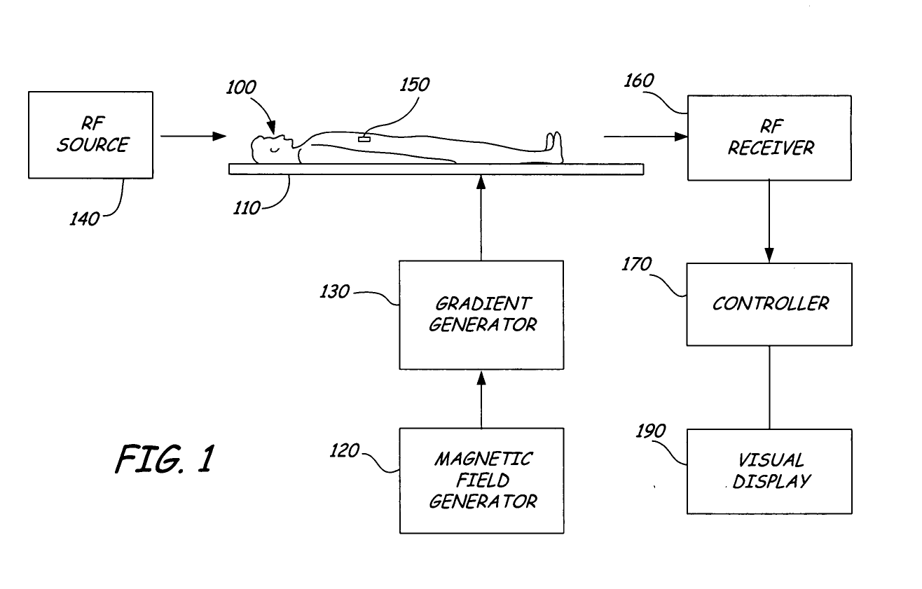

FIG. 1 is a partial block diagram of an illustrative magnetic resonance imaging system. In FIG. 1, subject 100 on support table 110 is placed in a homogeneous magnetic field generated by magnetic field generator 120. Magnetic field generator 120 typically comprises a cylindrical magnet adapted to receive subject 100. Magnetic field gradient generator 130 creates magnetic field gradients of predetermined strength in three mutually orthogonal directions at predetermined times. Magnetic field gradient generator 130 is illustratively comprised of a set of cylindrical coils concentrically positioned within magnetic field generator 120. A region of subject 100 into which a device 150, shown as a stent, has been inserted, is located in the body of subject 100.

RF source 140 radiates pulsed radio frequency energy into subject 100 and stent 150 at predetermined times and with sufficient power at a predetermined frequency to influence nuclear magnetic spins in a fashion known to those skilled in the art. The Larmor frequency for each spin is directly proportional to the absolute value of the magnetic field experienced by the atom. This field strength is the sum of the static magnetic field generated by magnetic field generator 120 and the local field generated by magnetic field gradient generator 130. In an illustrative embodiment, RF source 140 is a cylindrical external coil that surrounds the region of interest of subject 100. Such an external coil can have a diameter sufficient to encompass the entire subject 100. Other geometries, such as smaller cylinders specifically designed for imaging the head or an extremity can be used instead. Non-cylindrical external coils such as surface coils may alternatively be used.

External RF receiver 160 illustratively detects RF signals emitted by the subject in response to the radio frequency field created by RF source 140. In an illustrative embodiment, external RF receiver 160 is a cylindrical external coil that surrounds the region of interest of subject 100. Such an external coil can have a diameter sufficient to encompass the entire subject 100. Other geometries, such as smaller cylinders specifically designed for imaging the head or an extremity can be used instead. Non-cylindrical external coils, such as surface coils, may alternatively be used. External RF receiver 160 can share some or all of its structure with RF source 140 or can have a structure entirely independent of RF source 140. The region of sensitivity of RF receiver 160 is larger than that of the stent 150 and can encompass the entire subject 100 or a specific region of subject 100. The RF signals detected by external RF receiver 160 are sent to imaging and tracking controller unit 170 where they are analyzed. Controller 170 displays signals received by RF receiver 160 on visual display 190.

Establishing a homogenous, or uniform, magnetic field with magnetic field generator 120 in addition to switched linear gradient magnetic fields activated in various sequences as well as timely switching the RF radiowave in various sequences, as known in the art, enables the production of internal images of subject 100. It is common that stent materials and designs, in a configuration where there is an absence of RF artifacts, have limited effect on the magnetic fields generated by generator 120 and thus the stent is not easily detectable in MRI images. In accordance with an embodiment of the present invention, paramagnetic and/or ferromagnetic materials applied to the surface of and embedded into stent 150 will generally distort magnetic fields and provide a suitable marking that is visible in MRI images.

In one embodiment of the present invention, a support structure is configured such that magnetic field changes in a region immediately proximate the support structure, when induced by an MRI process, are substantially unobstructed by the support structure. The support structure may be at least partially metallic and configured such that there are no electrical loops or contain electrical loops oriented in the direction of the main magnetic field. For example, a single spiral conformation will not include electrical loops. Another configuration includes a floating_design wherein a plurality of open rings are connected to a single backbone that does not contain electrical loops. Suitable metallic materials include stainless steel, cooper, Nitinol, Tantalum, Titanium, Zirconium and/or combinations thereof.

The stent support structures may also include polymer or ceramic materials in addition to the metals described above. The polymeric and ceramic materials may be used in order to prevent electrical loops from forming in the support structure. For example, the support structure may be a braided structure having coated wires that prevent electrical contacts. The coated wires are arranged in a configuration that prevents electrical loops from forming in the support structure. Additionally, ceramic or polymeric materials may be used to connect metallic materials in the support structure such that electrical discontinuities are formed in the metallic structural members. Various configurations of stents that do not obstruct the MRI images are described in co-pending application “Medical Device with Magnetic Resonance Visibility of the Enhancing Structure,” Ser. No. 10/359,970, filed Feb. 6, 2003, and “Stent Designs Which Enable Visibility of the Inside of the Stent During MRI,” Ser. No. ______, filed ______, the contents of which are both hereby incorporated by reference.

Additional support structures that do not substantially obstruct magnetic field changes include polymers and ceramics, for example support structures made of ultra-high molecular weight polyethylene (UHMWPE) polyaryletherketone (PEEK) polymers such as PEEK-Optima, fiber reinforced polymers, flexible ceramics and/or combinations thereof. Additionally, support structures made of biodegradable materials such as polyvinyl alcohol (PVA) and polylactic acid (PLLA) may also be used.

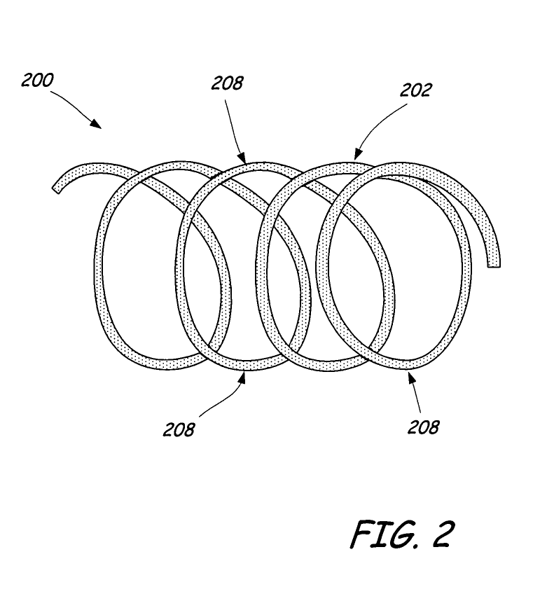

FIG. 2 is a view of a stent according to one embodiment of the present invention, which can be one embodiment of stent 150 in FIG. 1. Stent 200 includes a generally tubular structure 202 made up of a single band in a spiral conformation. The spiral conformation does not contain electrical loops, and therefore RF artifacts due to the effect of Faraday's Law are absent. The generally tubular structure 202 is adapted to frictionally engage an inner circumference of a lumen of a patient. Tubular structure 202 is made of a metallic, non-magnetic material having low magnetic susceptibility such as Nitinol. Other materials may also form tubular structure 202 including other metals, metallic alloys, polymers, ceramics and biodegradable materials.

Additionally, a magnetic material 208 has been applied to tubular structure 202. Magnetic material 208 may be a strong paramagnetic material such as dysprosium or terbium or a ferromagnetic material such as gadolinium, iron, manganese, nickel, cobalt and/or combinations thereof. For paramagnetic materials, in the presence of a magnetic field, there is a partial alignment of the atomic magnetic moments in the direction of the magnetic field, resulting in a net positive magnetization and positive magnetic susceptibility. When the magnetic field is removed, the net magnetic property is removed due to thermal vibrations. Ferromagnetic materials exhibit a substantially permanent magnetism even when a magnetic field surrounding the material is removed.

Magnetic material 208 is deposited on top of or embedded into tubular structure 202 or both. Enough magnetic material 208 is applied to tubular structure 202 such that tubular structure 202 is visible during an MRI procedure. The applied magnetic material 208 provides a significant disturbance in the surrounding magnetic field during an MRI procedure that is detectable by RF receiver 160. A suitable method to embed or deposit magnetic material 208 into tubular structure 202 is by using plasma immersion ion implantation (PIII). Other methods may also be used to apply magnetic material 208 to tubular structure 202 including crimping magnetic material 208 on the tubular structure 202.

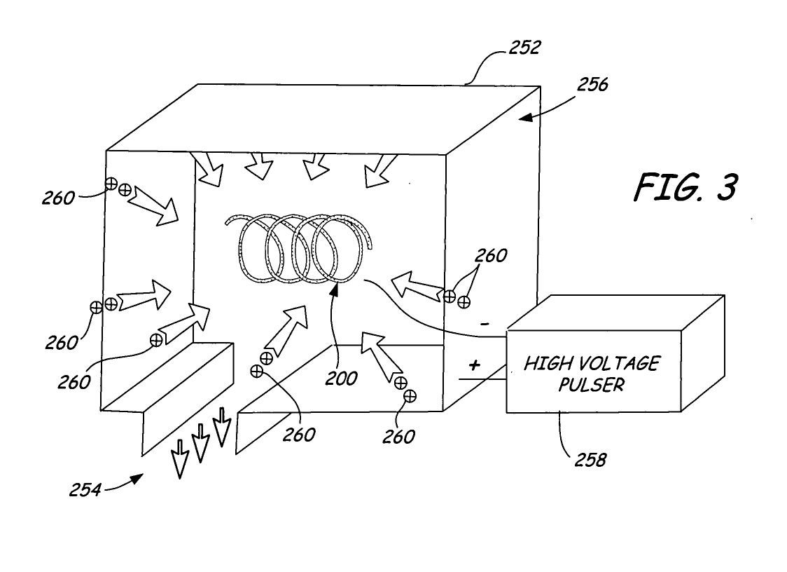

FIG. 3 illustrates an exemplary environment for performing PIII. In order to perform PIII, stent 200 is inserted into a chamber 252. Chamber 252 is a vacuum chamber created by vacuum 254 containing a plasma 256. Plasma 256 contains ions of a material (i.e. a paramagnetic or ferromagnetic material) to be implanted into stent 200. Stent 200 is pulsed repeatedly with high negative voltages from pulser 258. As a result of the pulses of negative voltages, electrons are repelled away from stent 200 and positive ions 260 are attracted to the negatively charged stent 200. As a result, positive ions strike all the surfaces of stent 200 and are embedded in and/or deposited onto stent 200.

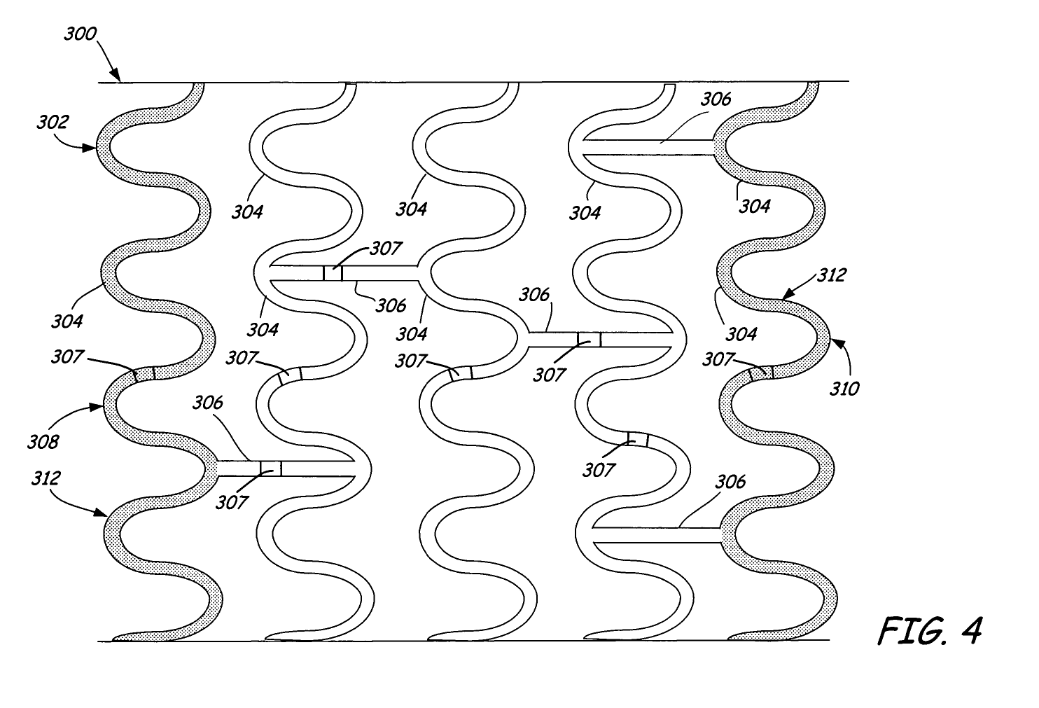

The magnetic material need not be applied to the entire tubular structure of the stent. FIG. 4 illustrates a stent 300 made of a tubular structure 302 having a plurality of rings 304 and a number of connectors 306 connecting the plurality of rings. A number of electrical discontinuities 307, which may be made of an insulating material, are provided throughout the tubular structure 302 to prevent electrical loops from forming in the tubular structure. An electrical discontinuity may be a joint or other connector that is configured to electrically isolate one electrically conductive segment from another electrically conductive segment. Exemplary discontinuities and designs not containing electrical loops are described in co-pending application, “Stent Designs Which Enable Visibility of the Inside of the Stent During MRI” referenced above. In this embodiment, only end portion 308 and end portion 310 include magnetic materials 312. The magnetic material can be applied to just a portion of stent 300 by using a shield during the plasma immersion ion implantation process.

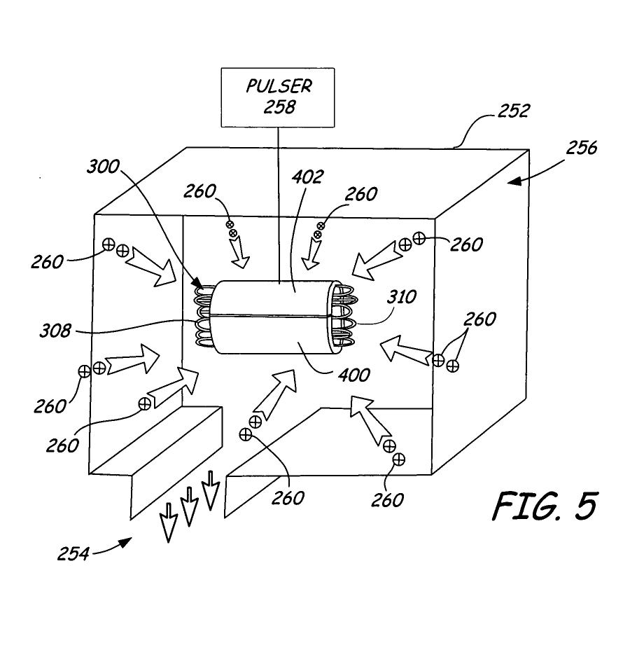

FIG. 5 illustrates an exemplary environment for providing PIII to a portion of stent 300. Stent 300 is clamped between two rings 400 and 402 that act as a shield such that end portion 308 and end portion 310 stick out of the rings 400 and 402. The rings provide a suitable clamp for stent 300. The rings 400 and 402 shield stent 300 so that ions from within chamber 252 are only applied to end portions 308 and 310. In one embodiment, metal rings may be used 400 and 402, wherein an electric contact is formed between rings and stent 300 in order to provide the negative voltage pulses. In this embodiment, pulser 258 is electrically coupled to rings 400 and 402. Other suitable materials such as a polymer may be used as rings 400 and 402.

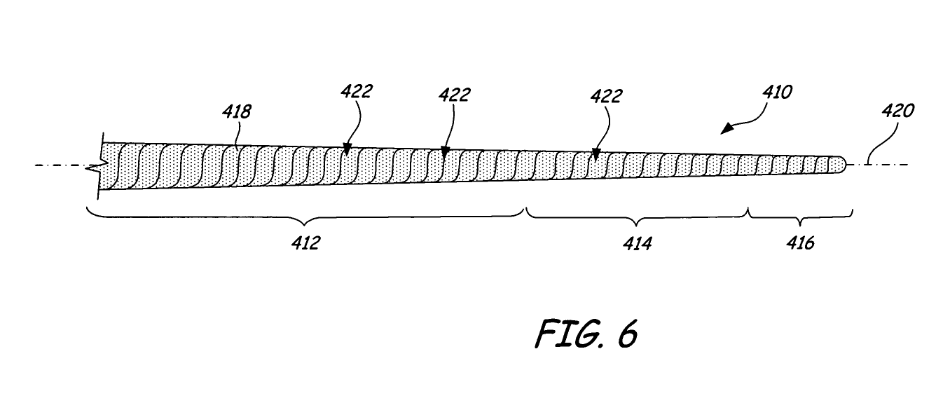

In order to provide markers for a polymer or ceramic guide wire, a magnetic material may be applied to the guide wire. FIG. 6 illustrates an exemplary guide wire 410. Guide wire 410 includes a relatively stiff proximal portion 412, a transition portion 414 with varying, intermediate stiffness and a highly flexible distal portion 416. The guide wire may be formed entirely of common medical or polymer materials and/or flexible ceramic materials. A segment 418 of wire that, prior to processing, was parallel to device axis 420, but after twisting and tensioning, follows a characteristic helical path provides high torque fidelity. The helical path of segment 418 is disposed about device axis 420. A magnetic material 422 is further applied to guide wire 410. The magnetic material 422 may be a paramagnetic or ferromagnetic material and applied to guide wire 410 or a portion thereof such that the guide wire is visible during an MRI procedure. An exemplary guide wire is described in U.S. Pat. No. 5,951,494, entitled “POLYMERIC IMPLEMENTS FOR TORQUE TRANSMISSION”, issued Sep. 14, 1999, the contents of which are hereby incorporated by reference.

By applying magnetic material to a support structure of a medical device, a significant disturbance is created in a surrounding magnetic field during an MRI procedure. The device may be formed of a variety of different materials and configured in a variety of different structures. As a result of the applied magnetic material, the support structure is detectable, and suitable monitoring of operation of the stent both during delivery and after deployment can be achieved. Many different methods can be used to apply the magnetic material to the support structure. A suitable way of applying magnetic material to the support structure is by using an implantation process such as plasma immersion ion implantation.

Although the present invention has been described with reference to illustrative embodiments, workers skilled in the art will recognize that changes may be made in form and detail without departing from the spirit and scope of the invention.

1. An implantable medical device, comprising:
a support structure formed such that magnetic field changes in a region immediately proximate the support structure, induced by a magnetic resonance imaging process, are substantially unobstructed by the support structure; and
a magnetic material coating at least part of the support structure.

2. The implantable medical device of claim 1 wherein the implantable medical device comprises a stent, the support structure forming a generally tubular structure that is substantially non-magnetic, and wherein the magnetic material coats the generally tubular structure such that at least part of the generally tubular structure is visible during a magnetic resonance imaging procedure.

3. The implantable medical device of claim 2 wherein the generally tubular structure is made of a metallic material.

4. The implantable medical device of claim 3 wherein the metallic material is at least one of Nitinol, stainless steel, tantalum, niobium, titanium and copper.

5. The implantable medical device of claim 2 wherein the generally tubular structure is made of at least one of a polymer and a ceramic.

6. The implantable medical device of claim 2 wherein the generally tubular structure is made of a biodegradable material.

7. The implantable medical device of claim 1 wherein the magnetic material is paramagnetic.

8. The implantable medical device of claim 1 wherein the magnetic material is ferromagnetic.

9. The implantable medical device of claim 1 wherein the magnetic material includes at least one of iron, dysprosium, gadolinium, terbium, copper, cobalt, manganese, chromium and nickel.

10. The implantable medical device of claim 2 wherein the generally tubular structure includes an end portion and the magnetic material is applied to the end portion.

11. The implantable medical device of claim 2 wherein the generally tubular structure includes a first end portion and a second end portion and the magnetic material is applied only to the first end portion and the second end portion.

12. A stent comprising:
a generally tubular structure made of a metallic material that is substantially non-magnetic; and
means for rendering the generally tubular structure visible during a magnetic resonance imaging procedure.

13. The stent of claim 12 wherein the metallic material is at least one of Nitinol, stainless steel, tantalum, niobium, titanium and copper.

14. The stent of claim 12 wherein the means for rendering is a paramagnetic material.

15. The stent of claim 12 wherein the means for rendering is a ferromagnetic material.

16. The stent of claim 12 wherein the means for rendering includes a material that is at least one of iron, dysprosium, gadolinium, terbium, copper, cobalt, manganese, chromium and nickel.

17. The stent of claim 12 wherein the generally tubular structure includes an end portion and the means for rendering is applied to the end portion.

18. The stent of claim 12 wherein the generally tubular structure includes a first end portion and a second end portion and the means for rendering is applied to the first end portion and the second end portion.

19. A method for making an implantable medical device, comprising:
forming a support structure such that magnetic field changes in a region immediately proximate the support structure, inducing by a magnetic resonance imaging process, are substantially unobstructed by the support structure; and
applying a magnetic material to at least part of the support structure such that the support structure is visible during a magnetic resonance imaging procedure.

20. The method of claim 19 wherein applying comprises using a plasma immersion ion implantation process.

21. The method of claim 20 wherein applying further comprises shielding a portion of the support structure so that the magnetic material is applied to only an end portion of the support structure.

22. The method of claim 20 wherein applying further comprises shielding the support structure so that the magnetic material is only applied to a first end portion and a second end portion of the support structure.

23. The method of claim 19 wherein the support structure is made of a metallic material.

24. The method of claim 23 wherein the metallic material is at least one of Nitinol, stainless steel, tantalum, niobium, titanium and copper.

25. The method of claim 19 wherein the support structure is made of at least one of a polymer and a ceramic.

26. The method of claim 19 wherein the support structure is made of a biodegradable material.

27. The method of claim 19 wherein the magnetic material is paramagnetic.

28. The method of claim 19 wherein the magnetic material is ferromagnetic.

29. The method of claim 19 wherein the magnetic material includes at least one of iron, dysprosium, gadolinium, terbium, copper, cobalt, manganese, chromium and nickel.

30. An elongated medical instrument comprising:
a support structure including a segment of material helically oriented about an axis of the instrument wherein the material is at least one of a polymer and a ceramic; and
a magnetic material applied to the segment.

31. The instrument of claim 30 wherein the magnetic material is paramagnetic.

32. The instrument of claim 30 wherein the magnetic material is ferromagnetic.

33. The instrument of claim 30 wherein the magnetic material includes at least one of iron, dysprosium, gadolinium, terbium, copper, cobalt, manganese, chromium and nickel.
